(19) United States
(12) Patent Application Publication
(10) Pub. No.: US 2010/0191201 A1
(43) Pub. Date: Jul. 29, 2010

(54) BODY WASTE COLLECTING DEVICE

(76) Inventors: Anders Bach, Kobenhavn (DK); Esben Stroebech, Hoersholm (DK); Mads Lykke, Broenshoej (DK)

Correspondence Address: JACOBSON HOLMAN PLLC, 400 SEVENTH STREET N.W., SUITE 600, WASHINGTON, DC, 20004, US

(21) Appl. No.: 12/452,225

(22) PCT Filed: Jun. 17, 2008
(86) PCT No.: PCT/DK2008/050143

§ 371 Date: Mar. 26, 2010

(30) Foreign Application Priority Data
Jun. 19, 2007 (DK) PA200700895

(51) Int. Cl. A61F 5/443; A61F 5/448

(52) U.S. Cl. 604/336; 604/344

(57) ABSTRACT

A collecting device comprising a collecting pouch and an adhesive wafer for attachment to the body, said wafer comprising at least one low-modulus backing layer and an elastic adhesive gel layer comprising a polyalkyleneoxide polymer and an organosiloxane based cross-linked adhesive system.

### BACKGROUND OF THE INVENTION

1. Field of the Invention

The invention relates to a collecting device for attachment to the body and for collecting bodily waste.

Collecting devices for collecting bodily waste, ostomy appliances, wound or fistulae drainage bandages or devices for collecting urine are usually in the form of a receptacle, e.g. a bag, pouch or tube for receiving the waste, connected to an adhesive wafer that can be attached to the skin of the patient. The wafer is typically in the form of a backing layer coated on the skin-facing surface with an adhesive layer and the wafer may further be provided with an aperture for accommodating the body opening. The size and shape of said aperture can often be adapted individually to fit the anatomy of the patient.

One of the crucial parts of such devices is the adhesive wafer. The wafer should be able to fit leak proof around the body opening and have good adherence to the skin without unintended detachment from the skin, but at the same time the wafer should be easy to remove again without damaging the skin. Furthermore, the wafer should be able to follow the movements of the body and be comfortable to wear. The components of the wafer, the adhesive and the backing layer determine these properties.

Pressure sensitive adhesives have for a long time been used for attaching medical devices, such as ostomy appliances, dressings (including wound dressings), wound drainage bandages, fistula drainage devices, devices for collecting urine, orthoses and prostheses to the skin.

The adhesive of such devices is usually a hydrocolloid adhesive coated in a relatively thick layer on a backing layer and combined with the fact that this adhesive is rather stiff, the device may be inflexible and bulky to wear.

Hydrocolloid adhesives containing hydrophilic particles or absorbents, which absorb moisture into the adhesive bulk and transmit moisture when conditions are saturated, are a well-known group of pressure sensitive adhesives useful for attaching medical devices to the skin. However, the retention of moisture in hydrocolloid adhesives may cause changes in the adhesive, such as swelling, loss of cohesion and disintegration. Non-absorbing adhesives on the other hand, may trap excessive moisture between the skin and the adhesive, causing weakening of adhesion and maceration of the skin.

Due to the delicate nature of skin, there is a narrow window where a pressure sensitive adhesive can function as a good and skin friendly adhesive: On one hand, the adhesive should be able to attach the medical device to the skin and the device should not fall of during wear and on the other hand, removal of the medical device from the skin should not cause damage to the skin.

Further, conventional pressure sensitive adhesives for collecting devices are usually based on adhesives that flow into the skin. This makes the adhesive very sticky to the skin, but also means that when the adhesive is removed, part of the top layer of the skin or epidermis is peeled of. This problem is not present for cross-linked adhesives, as they cannot flow into the skin.

For medical uses, a high water vapour transmission into the pressure sensitive adhesive is desirable. However, the availability of pressure sensitive adhesives with high water vapour transmission, which are suitable for skin contact use, is limited. Conventional absorbing pressure sensitive adhesives use high loads of absorbing particles in order to transport water into the adhesive, because of the low permeability of the polymer matrix. Using a more permeable polymer matrix reduces the need for high particle loading and a more soft and flexible adhesive can be obtained.

The water vapour transmitting pressure sensitive adhesives currently used for adhesion to the skin are mainly silicone and acrylate based adhesives.

Pressure sensitive adhesives based on acrylates are usually solvent based and may include toxic residues and monomers causing malodour. These adhesives may incorporate hydrophilic components, such as hydrocolloids, which absorb moisture. However, the content of hydrophilic components and hence the absorption of moisture change the properties of the adhesive, swelling of the adhesive and reduced adhesion being the most undesirable effects. Typically, the wear time of such acrylate adhesives is short due to the above-mentioned effects.

Silicone adhesives are relatively expensive and have a relatively low moisture transmission, which causes problems with regard to breathability. Adhesion may also be compromised when moisture is build up between the skin and the adhesive. Moreover, the compatibility of silicones with other organic materials (e.g. polymers) is limited, which affects the blending stability with performance enhancing additives as well as adhesion ability to reinforcement materials of other chemical compositions. Silicone adhesives are used for medical devices, especially wound dressings, but suffer from the drawback that they have a very low permeability to water. Adding hydrocolloids to these adhesives enhances the permeability, but renders the adhesive stiff. Alternatively a high permeability may be achieved by coating in a pattern, but this reduces the adhesive tack and increases the risk of leakage.

The backing layer of wafers for collecting devices is usually a polymer film. The backing layer used in conventional wafers is relatively rigid in that the adhesive in it self is stiff and rigid because of high particle loading and choice of polymer matrix. As the receptacle or coupling means for the receptacle usually are welded to the backing film, it is important that the backing layer is strong enough to handle this and the material chosen should be weldable as well.

2. Description of the Related Art

European Patent No. EP 1 424 088 discloses an ostomy device comprising a silicone adhesive. The adhesive is mainly intended for use for the coupling of the pouch to the wafer. Attachment to the skin is also mentioned, but the reference is silent with respect to choice of backing layer as well as the impermeability of silicone adhesive in skin contact.

International Patent application No. WO 2006/075948 discloses a component for making it easier to fasten a stoma bandage to the skin. The component is in the form of a disc comprising a plastic film coated with a layer of soft silicone elastomer, the disc is provided with a through-opening intended to be applied around a stoma. The component is intended to be used in combination with a standard stoma bandage, e.g. an adhesive wafer comprising hydrocolloid adhesive.

International Patent application No. WO 2006/075950 discloses a thin film dressing comprising a plastic film coated with a silicone adhesive.

Thus, there is still a need for a collecting device having a high flexibility and being comfortable for the user.

### SUMMARY OF THE INVENTION

The present invention aims at providing a body waste collecting device, which improves the patient's comfort due to the softness of the device and eliminates or—at least to a large extent—reduces the risk of skin irritation or skin damage, which may occur in the area around the body opening of a patient.

One object of the invention is to provide a soft and flexible attachment to the user's body.

Another object of the invention is to provide a collecting device with a skin-friendly adhesive, being easy and less painful to remove from the skin.

Yet another object of the present invention is to provide a device with good breathability, good adhesive tack and low risk of leakage.

### BRIEF DESCRIPTION OF THE DRAWINGS

The invention is disclosed more in detail with reference to the drawing wherein

FIG. 1 shows a preferred embodiment of the invention.

### DETAILED DESCRIPTION OF THE PRESENT INVENTION

The invention relates to a body waste collecting device comprising a collecting pouch and an adhesive wafer for attachment to the body, said wafer comprising at least one low-modulus backing layer and an elastic adhesive gel layer, wherein the adhesive layer comprises a polyalkyleneoxide polymer and an organosiloxane based cross-linked adhesive system.

By body waste collecting device is meant a device being able to collect and hold the output in a collecting item for a predefined time. The fixation of the device to the skin may be obtained by a skin adhesive and the collection may be obtained by a bag.

The use of a soft elastic gel type adhesive in a collecting device of the present invention provides completely new features to the user. Contrary to the traditional adhesive wafers comprising hydrocolloid adhesive, which is relatively stiff, the device according to the invention may provide the user with greater comfort as well as lower risk of leakage. It has surprisingly been shown that a device comprising such elastic gel adhesive in combination with a low-modulus backing layer provides an excellent attachment to the body.

It has surprisingly been found that the device according to the invention provides softness, flexibility, safety and comfort in wear and a good moisture transmission compared to devices comprising hydrocolloid adhesives.

Traditional ostomy appliances comprise an adhesive wafer, which is rather stiff. The stiffness derives from the properties of the adhesive, as well as those of the backing film. As the skin of the stomach is exposed to large movements in the form of stretching, flexing and folding during the user's movements it is important that the wafer is able to follow the movements of the skin.

By virtue of the fact that the adhesive layer of the device of the present invention is very soft, it can follow and adhere to irregularities in the skin so that fluid, which may leak from the opening, cannot pass underneath the adhesive wafer. The device according to the invention is also very shapeable, which means that the edge of the opening in the component can be applied very close to a stoma without risk of irritation, strangulation or bleeding of the mucous membrane at the base of the stoma.

The adhesive wafer of the device according to the invention can be stretched together with the skin in a way that there is considerably less risk of shearing between skin and adhesive, which shearing can give rise to mechanical damage to the skin and unintended detachment of the device.

A further advantage of the device according to the invention is that it is adherent to skin and can be reapplied after removal from skin, because it does not to any major extent tear off skin cells during removal, which would otherwise reduce the adherent surface of the component available for reapplication. Traditional hydrocolloid based adhesives, when removed, tear off so many skin cells that it is the surface area of the adhesive available for re-adhesions considerably decreased after detachment from the skin.

Yet another advantage of the device according to the described embodiment is that it maintains its integrity upon contact with fluid. In this context it should be noted that if the opening of the device is too small, it could be made larger by punching or cutting in order to adapt its size to the stoma. Conventional fastening arrangements for stoma bags are often provided with cutting marks, for example in the form of helical lines, to make this kind of adaptation easier. Such adaptation of size is important for ensuring that the smallest possible area of skin around the stoma comes into contact with the intestinal content collected in the stoma bag. As already mentioned, the shapeability of the device means that it is easy to finely adjust the shape of the opening, in a way that this coincides with the cross-sectional shape of the stoma, which may deviate from a circular shape.

Collecting devices are traditionally provided with hydrocolloid adhesive for attachment to the body. However, one of the drawbacks of hydrocolloid adhesives is their sensibility of erosion. When the hydrocolloid adhesive is exposed to moisture, the adhesive will swell and absorb the moisture. Unfortunately, the adhesion decreases during swelling and thus increases the risk of leakage. A high load of hydrocolloid is needed to facilitate permeability and absorption, but causes a relatively stiff product. The hydrocolloid adhesive is hard on the skin upon removal, as it peels off a layer of cells each time.

However, exchanging the hydrocolloid adhesive on the wafer of a collecting device with a soft gel adhesive is not an obvious thing to do. Using the same backing layer would result in a bad adhesion and the risk of the wafer detaching from the skin is high. Replacing the hydrocolloid adhesive with a soft gel adhesive will only be successful if the choice of backing layer is reconsidered and adapted to the new adhesive, rendering such replacement to be a more complicated process and not an obvious thing to do.

But changing the backing layer is not a simple thing to do either, it may influence other properties of the device, such as compatibility with the other components of the device, e.g. when welding, as well as on the permeability and flexibility of the device.

The backing layer of the device of the present invention is preferably in the form of a polymer film, coating, laminate, textile or non-woven. The backing layer is preferably a highly flexible film, being strong enough for attachment of e.g. couplings and/or pouch and for removing the device in one piece, but soft enough to follow the movements of the body.

A preferred backing layer is a polyurethane film.

Preferably, the backing layer has thermoplastic elements that enable welding of e.g. a pouch or coupling ring to the adhesive wafer. Preferred thickness of the backing layer is between 10-60 μm in order to maintain the softness of the adhesive wafer.

The device of the present invention is soft and comfortable to wear, having a good adhesive tack, but is yet easy and gentle to remove and is permeable to moisture, thus overcoming the drawbacks of the hydrocolloid adhesive devices. The gel adhesive is resistant to erosion and does not loose its tack when exposed to moisture.

The device of the present invention is soft, comfortable and pliable due to the unique combination of a gel adhesive comprising a polyalkyleneoxide polymer and organosiloxane based cross-linked adhesive system and a low modulus top.

The soft construction facilitates easy adaptation to scars, irregularities and skin-folds and low modulus of skin wafer. The device may be removed with minimal pain due to extreme flexibility and no skin cells are stripped off and thus no traumatisation of skin. The soft gel adhesive has a broad peel front and good tenacity during use. Reposition of adhesive is also possible without loss of tack. The adhesive is resistant to erosion and has a good water capacity due to the high water permeability and optionally use of mineral absorbers.

The adhesive of the invention has preferably a G* at 0.01 Hz less than 15000 Pa, preferably less than 7500 Pa as measured using the technique enclosed herein. This means that the adhesive is considerably softer than conventional adhesive systems used for attaching collecting devices to skin.

A soft backing layer is also preferred in order for the adhesive wafer to follow the movements of the body. The backing layer of the device according to the invention has preferably a force below 0.75 N/4 mm at 20% extension, preferably less than 0.5 N/4 mm, as measured using the technique described herein.

By low-modulus backing layer is meant a backing layer that has a force below 0.75 N/4 mm at 20% extension, preferably less than 0.5 N/4 mm, as measured using the technique described herein.

An important property of the device of the invention is that the adherence force of the soft gel adhesive used does not change with time or changes only to a small extent with time, during wear time of the device.

It is preferred that the entire skin-facing surface of the backing layer is coated with the elastic adhesive gel comprising a polyalkyleneoxide polymer and an organosiloxane based cross-linked adhesive system. Hereby, a soft wafer is achieved. In one embodiment of the invention the soft gel adhesive may only cover the peripheral part or the central part of the wafer. Such a wafer may have 10-90% of the total area covered by the soft adhesive system and the rest covered by conventional ostomy type adhesives.

The elastic adhesive gel may comprise a layer of low-absorbent adhesive. The adhesive layer may be in the form of a laminate of two or more adhesives with different properties. By different properties is meant e.g. absorption, permeability or mechanical properties. The first adhesive layer may be absorbent while the second may be low-absorbent. The absorbency of the adhesive may be achieved by incorporating absorbent material in the adhesive, e.g. in the form of absorbent particles or salt.

It is preferred that the low-absorbent adhesive layer is on the skin-facing surface. Having a thin layer of low-absorbent adhesive facing the skin, combined with another layer of absorbent adhesive facing the backing layer, provides a skin-friendly attachment to the skin being capable of transporting moisture away from the skin and into the absorbing layer.

By low-absorbent is meant that the water absorption capacity is less than 8%, preferably less than 4%, as defined herein.

The adhesive used in the device of the present invention has a high moisture vapour transmission rate of the continuous polymer phase, preferably a MVTR over 100 g/m²/24 hrs as defined herein, which makes it breathable and very skin friendly. The high moisture transmission of the adhesive is a particular advantage, where a medical device has to be worn on the skin for a long time, e.g. days.

As used herein a cross-link means a small region in a macromolecule (polymer chain structure) from which more than 2 chains emanate.

The adhesive layer of the device of the invention comprises a polyalkyleneoxide polymer and an organosiloxane based cross-linked adhesive system.

According to one embodiment of the invention, the adhesive layer of the device comprises the reaction product of:

(i) a polyalkyleneoxide polymer having one or more unsaturated end groups and

(ii) an organosiloxane comprising one or more Si—H groups, carried out in the presence of an addition reaction catalyst.

According to another embodiment of the invention, the pressure sensitive adhesive composition of the device comprises more than 90% w/w of the polyalkylene oxide polymer that consists of polymerised alkyleneoxide moities having three or more carbon atoms.

According to another embodiment of the invention, the adhesive composition of the device comprises the reaction product of:

(i) a polyalkyleneoxide polymer having at least two unsaturated end groups and wherein more than 90% w/w of the polyalkylene oxide polymer consists of polymerised alkyleneoxide moities having three or more carbon atoms,

(ii) a polysiloxane cross-linking agent comprising 3 or more Si—H groups and optionally

(iii) a polysiloxane chain extender comprising up to 2 Si—H groups carried out in the presence of an addition reaction catalyst.

According to a preferred embodiment of the invention, the addition reaction catalyst is a Pt vinyl siloxane complex.

According to a preferred embodiment of the invention, the polyalkylene oxide polymer is polypropyleneoxide.

According to a further preferred embodiment of the invention, the weight percent of polyalkylene oxide in said reaction product is 60% or above.

The polyalkylene oxide polymer having one or more unsaturated groups may be branched or linear.

However, suitably, the polyalkylene oxide polymer is linear and has two unsaturated end groups.

In one particular embodiment of the invention, the polyalkylene oxide polymer is polypropyleneoxide.

The polypropylene oxide having unsaturated end groups may be a compound of formula

CH₂═C(R¹)-(Z)-O—(X)ₙ—(W)—C(R²)═CH₂ (Ia)

or

CH(R¹)═CH-(Z)-O—(X)ₙ—(W)—CH═CH(R²) (Ib)

wherein R¹ and R² are independently selected from hydrogen and C₁₋₆-alkyl;
Z and W is C₁₋₄-alkylene;

### X is —(CH₂)₃—O— or —CH₂—CH(CH₃)—O—; and

n is 1-900, more preferred 10-600, or most preferred 20-600.

The number average molecular weight of the polyalkylene oxide having unsaturated end groups is suitably between 500 and 100.000, more preferred between 500 and 50.000 and most preferred between 1.000 and 35.000.

Polypropylene oxide having unsaturated end groups may be prepared as described in U.S. Pat. No. 6,248,915 and WO No. 05/032401 or analogously to the methods described therein. Other polyalkylene oxide polymers may be prepared analogously.

The polysiloxane cross-linking agent comprising 3 or more Si—H groups is suitable a compound having the formula

R—SiO(R,R)—(SiO(R,R))ₘ—Si—(R,R,R) (II)

wherein at least three of the groups R are hydrogen and the rest of the groups R are each independently selected from C₁₋₁₂-alkyl, C₃₋₈-cycloalkyl, C₆₋₁₄-aryl, and C₇₋₁₂-arylalkyl; and m is 5-50, or preferably 10-40. The number average molecular weight as determined by GPC is suitably 500-3.000.

One or more cross-linking agents of formula (II) may be used in the cross-linking reaction.

In one embodiment of the invention, a mixture of one or more cross-linking agents of formula (II) comprising 3 or more Si—H groups and a polysiloxane chain extender comprising up to 2 Si—H groups is used in the cross-linking reaction.

The polysiloxane chain extender is suitably a compound having the formula

R³—SiO(R³,R³)—(SiO(R³,R³))ₘ—Si—(R³,R³,R³) (III)

wherein up to 2 of the groups R³ are hydrogen and the rest of the groups R³ are each independently selected from C₁₋₁₂-alkyl, C₃₋₈-cycloalkyl, C₆₋₁₄-aryl, and C₇₋₁₂-arylalkyl; and m is 0-50. The number average molecular weight as determined by GPC is suitably between 200 and 65.000, most preferably between 200 and 17.500.

As used herein C₁₋₁₂-alkyl means a linear or branched alkyl group having 1 to 12 carbon atoms, C₁₋₈-alkyl means a linear or branched alkyl group having 1 to 8 carbon atoms, and C₁₋₆-alkyl means a linear or branched alkyl group having 1 to 6 carbon atoms, such as methyl, ethyl, propyl, isopropyl, butyl, pentyl and hexyl.

As used herein C₁₋₄-alkylene means a linear or branched divalent alkylene group having 1 to 4 carbon atoms, such as methylene, ethylene, propylene, isopropylene, butylenes and isobutylene.

As used herein C₃₋₈-cycloalkyl means a cyclic alkyl group having 3-8 carbon atoms, such as cyclopentyl and cyclohexyl.

As used herein C₆₋₁₄-aryl means a phenyl or naphthyl group optionally substituted with C₁₋₆-alkyl, such as tolyl and xylyl.

As used herein C₇₋₁₂-arylalkyl means aryl attached to a C₁₋₆-alkyl group, where C₁₋₆-alkyl and aryl is as defined above, such as benzyl, phenethyl and o-methylphenethyl.

In the compound of formula (II) and in the compound of formula (III), the groups R and R³, which are not hydrogen, are suitably each independently selected from a member of the group C₁₋₆-alkyl, C₆₋₁₄-aryl or C₇₋₁₂-arylalkyl.

The Si—H groups may be situated at either end of the compound of formula (II). However, at least one Si—H group is preferably positioned within the —(SiO(R³,R³))ₘ— chain of the compound of formula (II).

The polysiloxane cross-linking agent and the chain extender may be prepared as described in Japanese Patent Application No. 2002-224706 and WO No. 05/032401 or analogously to the methods described therein.

An addition reaction is, in its simplest terms, a chemical reaction in which the atoms of an element or compound react with a double bond or triple bond in an organic compound by opening up one of the bonds and becoming attached to it, forming one larger compound. Addition reactions are limited to chemical compounds that have multiple-bonded atoms. Hydrosilylation is an addition reaction between, for example, a carbon-carbon double bond in a compound and a reactive hydrogen from a hydrogen siloxane.

Suitable addition reaction catalysts are any hydrosilylation catalysts, preferably platinum (Pt) catalysts. Pt-catalysts for the first part of the two-component sealant are described in U.S. Pat. No. 6,248,915. In consideration of toxicity potential, Pt complex catalyst where Pt is at a valency state of zero is preferred. Preferred catalysts are platinum-vinylsiloxanes and platinum-olefin complexes, such as Pt-divinyl tetramethyl disiloxane.

The reaction is suitably carried out neat at a temperature between 25° C. and 150° C. It is not necessary to use a solvent for the reaction, which is an advantage for any adhesive, but especially for skin applications.

Suitably, the ratio of the number of reactive Si—H groups in the polysiloxane cross-linking agent to the number of unsaturated groups in the polypropylene oxide, which are reactive with Si—H groups under the reaction conditions, is between 0.2 and 1.0.

The amount of polysiloxane used for the cross-linking is suitably less than 15% w/w and more preferred below 10% w/w of the amount of polyalkylene oxide polymer having unsaturated end groups.

The cross-linking reaction does not lead to complete cross-linking of all the polyalkylene oxide polymers. The adhesive comprises a mixture of cross-linked and non cross-linked polyalkylene oxide polymer.

The pressure sensitive adhesive composition of the device according to the invention may contain other conventional ingredients for adhesive compositions, such as tackifiers, extenders, non-reactive polymers, oils (e.g. polypropylenoxide, ethyleneoxide-propyleneoxide copolymers, mineral oil), plastizisers, fillers, and surfactants. The adhesive may also comprise pharmaceutically active ingredients. These optional ingredients may be present in the reaction mixture during the cross linking reaction.

It may be advantageous that the elastic adhesive gel comprises absorbent particles. The particles may be absorbent articles such as mineral salt, hydrocolloid or super absorbers in order for the adhesive to absorb moisture from skin.

Preferred particle size of the absorbent particles is smaller particles, as they are more difficult to see by the naked eye and will give products that are more pleasing to the eye. An upper limit on particle size is the size of the smallest dimension of the adhesive. Thus, a 300 μm thick adhesive should not contain particles with diameters above 300 μm. There is a tendency of the hygroscopic particles to agglomerate and this effect will increase with decreasing particle size.

Therefore, a preferred particle size would be from 10-300 μm. Also, the particles may contain an anti agglomerating agent to reduce agglomeration of small particles.

Microcolloid particles may also be incorporated. Microcolloid particles are well known in the art e.g. from International Patent Application No. WO 02/066087, which discloses adhesive compositions comprising microcolloid particles. The microcolloid particles may have a particle size of less than 20 microns.

Salt may be advantageous to use as absorber if it is contained within an ion impermeable matrix like the hydrophobic adhesive used in the device of this invention. Some salts like sodium chloride have an equilibrium vapour pressure of about 75% at skin temperature and will absorb water from skin and output because of the difference in vapour pressure.

In a preferred embodiment of the invention, the adhesive comprises particles of mineral salt. The salt may be present in an amount of 1-50% w/w, more preferred in an amount of 5-30%.

In one embodiment of the invention, the adhesive comprises non-absorbent particles which presence may modify the rheologic properties of the adhesive.

The absorbent adhesive layer may comprise 1-40% w/w of hydrocolloid (HC) or super absorbent particles (SAP) particles, more preferred 5-30% w/w particles.

The device of the present invention may have an absorbency of the adhesive of 0.01-0.1 g/cm² more preferred 0.01-0.75 g/cm² as measured using the method enclosed herein.

The collecting pouch may be detachable from the adhesive wafer by a coupling system or the pouch and the wafer may be integrated with the wafer, e.g. by welding. The two versions are known as one piece or two-piece appliances for ostomy.

In order to avoid rolling up of the edge portion during wear, it may be advantageous to bevel the edge portion of the wafer.

According to an embodiment of the invention, the collecting device is an ostomy appliance.

According to another embodiment of the invention, the collecting device is a faecal collecting device.

According to another embodiment of the invention, the collecting device is a fistula collecting device.

### DESCRIPTION OF THE PREFERRED EMBODIMENTS

The invention is now explained more in detail with reference to the drawings showing preferred embodiments of the invention.

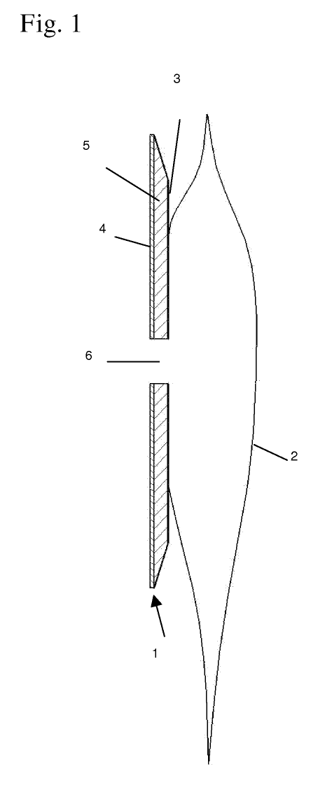

In FIG. 1 is shown a preferred embodiment of the invention. The device comprises an adhesive wafer (1) onto which is mounted a collection pouch (2) for receiving bodily waste. The wafer comprises a backing layer (3), to which the pouch (2) is attached, either by welding or by coupling means allowing detachment and change of the pouch (2) without removing the wafer (1) from the skin. On the skin-facing surface of the backing layer is a layer of adhesive containing salt particles (5) and the skin-facing surface of this layer is provided with a layer of low-absorbent adhesive (4) for attachment to the skin. The wafer is provided with a central aperture (6) for accommodating a body opening such as a stoma. The adhesive surface may further be provided with a release liner (not shown) to be removed before application.

### Materials and Methods

Determination of Moisture Vapour Transmission Rate (MVTR)

MVTR was measured in grams per square meter (g/m²) over a 24 hours period using an inverted cup method. A container or cup that is water and water vapour impermeable and having an opening was used. 20 ml saline water (0.9% NaCl in demineralised water) was placed in the container and the opening was sealed with the test adhesive in the form of a film sheet. The container, with a duplicate, was placed into an electrically heated humidity cabinet and the container or cup was placed upside down in a way that the water was in contact with the adhesive. The cabinet was maintained at 37° C. and 15% relative humidity (RH). After about an hour, the containers were considered to be in equilibrium with the surroundings and were weighed. 24 h after the first weighing, the containers were weighed again. The difference in weight is due to evaporation of vapour transmitted through the adhesive film. This difference was used to calculate the moisture vapour transmission rate or MVTR. The MVTR was calculated as the weight loss after 24 h divided by the area of the opening in the cup (g/m2/24 h). The MVTR of a material is a linear function of the thickness of the material. Thus, when reporting MVTR to characterise a material, it is important to inform the thickness of the material to which MVTR is reported. We used 150 μm as a reference and all MVTR measurements should be performed on polymer films with this thickness.

Determination of Water Absorption

Pieces of adhesive of 1×25×25 mm³ were fastened on a piece of glass using double sided adhesive and the constructs were immersed in saline water (0.9% NaCl in demineralised water) at 37° C. The samples were removed and carefully dripped dry and weighed after 2 hours. The change in weight was recorded and reported as weight gain in g/cm². Alternatively, the change in weight was recorded and reported as weight gain in percent of the original dry weight of the adhesive.

### Determination of Softness of Backing Layer

For measuring softness of the adhesive wafer, the testing guidelines from standard ISO527-1 were used. However, the parameters defined in ISO527-1 are in it self not sufficient to exactly describe the relevant parameters for ostomy devices. An ostomy device is placed on the stomach, on skin that can easily deform more than 20%. The relevant deformation for a soft adhesive wafer with a soft backing is in the same magnitude and we have therefore defined softness (modulus) of adhesive wafers as the force in Newton at 20% deformation divided by initial sample width. We used ‘dog-bone’ test specimens similar to the ones described in ISO 527-2 FIG. 1, but with different dimensions to accommodate the fact that some adhesive wafers are too small to be tested with ISO 527-1. We used test samples that scale with the samples from ISO527.2 FIG. 1, but where the width b₁ of the narrow portion was 4 mm and Gauge length L₀ was 10 mm. Relative deformation ε was calculated as the absolute deformation ΔL divided by the initial length L₀ as described in ISO 527-1. The rate of deformation was set to 1 mm/s. To accommodate for the fact that most films are isotropic, samples were measured in the softest direction. The obtained values are averages of at least 3 measurements.

Determination of G*

The parameter G* or complex modulus as defined in “Dynamics of polymeric liquids”, Vol. 1, sec. ed. 1987, Bird, Armstrong and Hassager, John Wiley and Sons inc., was used as a measure of the hardness of an adhesive. G* at 32° C. and 0.01 Hz was measured as follows: A plate of un-foamed adhesive material was pressed into a plate of 1 mm thickness. A round sample of 25 mm in diameter was cut out and placed in a RheoStress RS600 rheometer from Thermo Electron. The geometry applied was parallel plates 25 mm and the deformation was fixed at 1% to ensure that measurements were in the linear regime. The measurement was carried out at 32° C.

### EXAMPLES

The following materials were used to prepare a soft elastic adhesive gel collecting device according to the invention:

ACX003, allyl-terminated polyether (poly propylene oxide) viscosity 16 Pa·s from Kaneka.
Catalyst, Pt-VTS. Pt-VTS is Pt-divinyl teteramethyl disiloxane in IPA (Pt 3.0 wt %).
CR600, poly-alkyl hydrogen siloxane curing agents available from Kaneka.
NaCl, Salt, 99.9% NaCl from Sigama Aldrich.
Polyurethane film, Bioflex 130, 25 my form Scapa.
Barrier film for collecting device from DOW, Saranex 650.Super absorber
Luquasorb from BASF

### Example 1

100 g of adhesive base was produced by mixing polymer AC003, cross-linker CR600 and catalyst in the ratios (w/w) given in Table 1.

TABLE 1

| | |
|---|---|
| Polymer AC003 | 96.55 |
| Cross-linker CR600 | 3.35 |
| Catalyst | 0.10 |

25 g of super absorber was mixed into the adhesive in a way that the continuous adhesive phase was 80% w/w and the absorbing discontinuous phase was 20% w/w. The soft elastic adhesive wafer was produced by pouring approximately 10 g of the liquid pre-mixture onto a polyurethane film covered mould of a diameter of 100 mm and a thickness of 1 mm. The excess liquid mixture was removed by scraping. A releasable protective film was applied on the top of the liquid adhesive mixture and the mould with all the contents were placed in an oven for 1 hour at 100° C. for curing. After curing the adhesive wafer was die cut into a diameter of 99 mm and barrier films forming a pouch were heat welded to the adhesive wafer by conventional means (2 sec, 4 bar, 160° C.) giving the collecting device according to the invention. In order to get a perfect weld, a ring of a barrier film and polyurethane backing layer compatible film were placed between the barrier film and the backing layer of the adhesive wafer before welding.

### Example 2

100 g of adhesive base was produced by mixing polymer AC003, cross-linker CR600 and catalyst in ratios given in Table 1.

10 g of this mixture was distributed on a release liner in 100 μm thickness by scraping and the film was cured in an oven for 10 min at 100° C. 12.5 g of salt was added to 50 g of the adhesive mixture in a way that the continuous adhesive phase was 80% w/w and the absorbing discontinuous salt phase was 20% w/w. The soft elastic adhesive wafer was produced by pouring approximately 10 g of this liquid mixture onto a polyurethane film covered mould of a diameter of 100 mm and a thickness of 0.9 mm. The excess liquid adhesive mixture was removed by scraping. Then the cured 100 my adhesive layer was placed on top of the liquid mixture containing salt and the construction was cured for 60 min at 100° C. and converted into a collection device (as in Example 1). In this way the adhesive wafer was a layered structure with a 100 μm non-absorbing skin-facing layer, a soft backing layer and in between an absorbing layer containing 20% w/w salt.

G*, water absorption, MVTR and softness were determined as described above. The results are shown in Table 2.

TABLE 2

| | Example 1 | Example 2 |
|---|---|---|
| G* at 0.01 Hz for adhesive [Pa] | 2000 | 2700 |
| Water absorption of absorbing | 0.02 | 0.05 |
| adhesive after 2 h [g/cm2] | | |
| MVTR of continuous adhesive | 1200 | 1200 |
| phase [g/m2/24 h] | | |
| Softness of backing layer [N/4 mm] | 0.43 | 0.43 |

1. A body waste collecting device comprising a collecting pouch and adhesive wafer for attachment to the body, said wafer comprising at least one low-modulus backing layer and an elastic adhesive gel layer comprising a polyalkyleneoxide polymer and an organosiloxane based cross-linked adhesive system.

2. The device according to claim 1, wherein the polyalkylene oxide polymer is polypropyleneoxide.

3. The device according to claim 1, wherein G* of the adhesive is less than 15.000 Pa.

4. The device according to claim 1, wherein G* of the adhesive is less than 7.500 Pa.

5. The device according to claim 1, wherein the elastic adhesive gel covers the entire skin-facing surface of the backing layer.

6. The device according to claim 1, wherein the elastic adhesive gel comprises a layer of low-absorbent adhesive.

7. The device according to claim 6, wherein the low-absorbent adhesive layer is on the skin-facing surface.

8. The device according to claim 1, wherein the elastic adhesive gel layer comprises at least one layer of an absorbent adhesive.

9. The device according to claim 1, wherein the elastic adhesive gel comprises absorbent particles.

10. The device according to claim 9, wherein the absorbent particles are salt.

11. The device according to claim 9, wherein the absorbent particles are hydrocolloids.

12. The device according to claim 9, wherein the absorbent particles are microcolloids.

13. The device according to claim 9, wherein the absorbent particles are super absorbent particles.

14. The device according to claim 10, wherein the absorbent adhesive layer comprises 1-50% w/w of salt.

15. The device according to claim 11, wherein the absorbent adhesive layer comprises 1-40% of super absorbent particles, microcolloids or hydrocolloids.

16. The device according to claim 1, wherein the absorbency of the elastic adhesive layer is 0.02-0.4 g/2 h.

17. The device according to claim 1, wherein the absorbency of the elastic adhesive layer is 0.05-0.25 g/2 h.

18. The device according to claim 1, wherein the permeability of elastic adhesive gel is above 100 g/m²/24 h.

19. The device according to claim 1, wherein the permeability of elastic adhesive gel is above 200 g/m²/24 h.

20. The device according to claim 1, wherein the low-modulus backing layer is in the form of a polymer film, coating, laminate, textile or non-woven.

21. The device according to claim 1, wherein the low-modulus backing layer has a force below 0.75 N/4 mm at 20% extension, preferably less than 0.5 N/4 mm.

22. The device according to claim 1, wherein the collecting pouch is detachable.

23. The device according to claim 1, wherein the collecting pouch is integrated with the wafer.

24. The device according to claim 1, wherein the elastic adhesive gel layer has a thickness between 0.3 mm and 2.5 mm.

25. The device according to claim 1, wherein the elastic adhesive gel layer has a thickness between 0.5 mm and 1.5 mm.

26. The device according to claim 1, wherein the collecting device is an ostomy appliance.

27. The device according to claim 1, wherein the wafer has 10-90% of total area covered by the soft adhesive system.

28. The device according to claim 1, wherein the adhesive layer comprises one or more active agents.

29. The device according to claim 1, wherein the collecting device is an ostomy appliance.

30. The device according to claim 1, wherein the collecting device is a faecal collecting device.

31. The device according to claim 1, wherein the collecting device is a fistula collecting device.
